(19) United States
(12) Patent Application Publication
(10) Pub. No.: US 2004/0126422 A1
(43) Pub. Date: Jul. 1, 2004

(54) Novel co-processing method for oral drug delivery

(76) Inventors: Cheng Der Tony Yu, Baltimore, MD (US); Ngoc Do, Joppatowne, MD (US); Larry Augsburger, Severna Park, MD (US)

Correspondence Address: UPM PHARMACEUTICALS INC., 6200 SEAFORTH STREET, BALTIMORE, MD, 21224, US

(21) Appl. No.: 10/444,621

(22) Filed: May 23, 2003

Related U.S. Application Data
(60) Provisional application No. 60/437,507, filed on Dec. 31, 2002.

(51) Int. Cl. A61K 9/20; D04H 1/00

(52) U.S. Cl. 424/465; 264/109

(57) ABSTRACT

The new process can be summarized in five general phases: the manufacture and coating of active-loaded beads with a sustained release coat, an enteric coat; a colonic coat, or a taste-masking coat; the manufacture of the cushioning components the co-processing of active-loaded beads with cushioning components into a single component known as a Cushioning Bead™; the use of freeze-drying to produce the intended outcome, and the compression of the resultant Cushioning Beads™ into tablets.

### PRIORITY

This application is a Continuation-In-Part of Application 60/437,507 filed on Dec. 31, 2002. This application claims priority back to Application 60/437,507 and incorporates said application by reference.

### BACKGROUND

The ability to produce Cushioning Beads™ that incorporate an active-loaded bead on a large scale presented a number of manufacturing difficulties. The described co-processing method overcomes the large scale manufacturing difficulties and ensures that the process is optimized for today's drug manufacturing facility.

The previous work related to Cushioning Beads™ was discussed in U.S. Pat. No. 5,780,055. In the referenced patent, the Cushioning Beads™ were formed separately and then added to the active-loaded beads to create a mixture that was then compressed into a tablet. However, the technology was impractical for large scale manufacturing because of unexpected problems encountered when conventional production equipment was used. These problems include segregation of the Cushioning Beads™ from the active-loaded beads due to their size and density differences and the lack of dose uniformity due to beads segregation. The segregation problem can occur during transport of the mixture of the beads and in the hopper prior to tableting. The dose uniformity problem occurs when beads segregate.

The current invention will describe in detail the manufacturing process necessary to produce co-processed Cushioning Beads™ and active-loaded beads thereby producing a mixture that will allow for large scale production and the accommodation of multiple active ingredients in the active-loaded beads.

### DETAILED DESCRIPTION OF THE INVENTION

The current invention describes a new process which overcomes the segregation and the dose-uniformity problems during manufacture and therefore enables the use of conventional manufacturing equipment for the product.

The process, according to this invention, allows the active and the cushioning components to be made into a single component at the ratio of active to cushioning ranging from 0.1% to 97%. The resultant granules, which consist of the active beads embedded within a layer of porous cushioning material, provide protection for the active-loaded beads during compression, see FIG. 1. Furthermore, the new process allows a uniform distribution of the active-loaded beads throughout the cushioning material and prevents any segregation of the two components during transport or in the hopper. The dose uniformity of the tablets is assured, as a result. Finally, the new process will allow for multiple active pharmaceutical ingredients to be incorporated into the active-loaded beads. In the case of incompatible active pharmaceutical ingredients, the new process also allows the mix and match of separately made single active loaded beads mixed with the cushioning component to achieve Cushioning Beads™ that contain multiple actives.

The new process can be summarized in five general phases: the manufacture and coating of active-loaded beads with a sustained release coat, an enteric coat; a colonic coat, or a taste-masking coat; the manufacture of the cushioning components the co-processing of active-loaded beads with cushioning components into a single component known as a Cushioning Bead™; the use of freeze-drying to produce the intended outcome, and the compression of the resultant Cushioning Beads™ into tablets (see FIG. 2).

Manufacture and Coating of Active-Loaded Beads with a Sustained Release Coat, an Enteric Coat, a Colonic Coat, or a Taste-Masking Coat

Biologically active ingredients are contained in the active-loaded beads. The configuration of the active-loaded beads can be either a matrix, in which the biologically active ingredients are distributed throughout the inactive pharmaceutical excipients, or a drug-layered bead, in which layers of the biologically active ingredients are deposited around an inert nonpareil seed. In addition, the active-loaded bead can contain more than one active pharmaceutical ingredient. For the former, an extrusion/spheronization process is employed. A moistened, well-mixed mass of active and inactive ingredients is extruded into strands and subsequently rounded into spheroids or pellets in a spheronizer and dried in an oven or a fluid-bed dryer. A typical formulation for extrusion-spheronization consists of microcrystalline cellulose in combination with lactose, starch and other appropriate pharmaceutical excipients. For the latter, the biologically active ingredients are dispersed in a binder solution that can be layered onto nonpareil seeds using a typical fluid-bed coater. The binder solution includes but not limited to low-viscosity hydroxypropyl-methylcellulose.

The active-loaded beads are further coated with functional polymers to achieve a sustained-release delivery, an enteric delivery, a colonic delivery, or taste masking. Different polymers are used, pending the objective of the drug delivery. Two classes of polymers are commonly used for a sustained release coating, cellulosic polymer and methacrylate ester copolymer. Examples of these polymers are Eudragitg® NE, Eudragit® RS/RL, Aquacoat® and Surelease®. Other excipients such as plasticizer, secondary polymers, water-soluble and water-insoluble additives are often included in the formulation to achieve a desired dissolution profile. Enteric polymers are employed to prevent the contact of the biologically active ingredients with gastric juice and to facilitate the release of the drug in the small intestine region of the GI tract. Examples of the enteric polymers are Eudragit® L and S, Aquateric® and Sureteric®.

Manufacture of the Cushioning Component

The cushioning component consists of a highly-compactable filler, such as microcrystalline cellulose, in combination with a highly water-absorbing material, such as Ac-Di-Sol®. Disintegrants and superdisintegrants, such as starch, croscarmellose sodium, crospovidone, and sodium starch glycolate, or hydrophilic materials, such as hydroxypropyl cellulose, can be used as the highly water-absorbing material. The highly water-absorbable materials can range of 5 to 90% (w/w).

The cushioning components are dry-blended and then granulated in a planetary mixer via a typical low-shear wet granulation process with purified water as a granulating fluid. The cushioning compontents reaches the end-point once granules are produced by visual inspection.

Co-Processing of Cushioning Components and Active-Loaded Beads

Sustained-release coated, colonic coated, enteric coated, or taste-masking coated active-loaded beads are subsequently added to the cushioning components to produce the Cushioning Beads™. The active-loaded beads can alternatively be added to the cushioning dry-powder blend prior to the wet granulation step. The moistened granules of the well dispersed, active-loaded beads in the cushioning components are either passed through screen of appropriate size or extruded and spheronized. The beads or pellets thus obtained are then freeze-dried.

Freeze-Drying of the Cushioning Beads™

The co-processed Cushioning Beads™ are then placed into a freeze-dryer until a Loss on Drying (LOD) of less than 5% is achieved. Upon achievement of the LOD, the Cushioning Beads™ can be placed through a sieve to remove fines or move directly to the tableting process.

The freeze-drying creates the unexpected cushioning characteristic of the beads or pellets and produces a very porous layer which surrounds the active-loaded beads. Protected by the high porosity cushioning layer, the coatings of the active-loaded beads can withstand the compression force during normal tableting process. In addition, to the cushioning characteristic, the freeze-drying creates a non-hygroscopic Cushioning Bead™ that does not require any special handling or packaging.

Compression of the Freeze-Dried Cushioning Beads™ into Tablets

The compression of the final product, the Cushioning Bead™, follows a normal tablet compression operation. An additional advantage of the current invention is that no additional extra-granular ingredient, especially the binder, is required, because of the inter-locking mechanism created by the deformation of the cushioning layer during compression. The resultant tablets not only can maintain their mechanical strength but also can disintegrate rapidly upon contact with water, in less than 10 seconds depending on the amount of active.

This invention also encompasses prodrug derivatives of the compounds contained herein. The term “prodrug” refers to a pharmacologically inactive derivative of a parent drug molecule that requires biotransformation, either spontaneous or enzymatic, within the organism to release the active drug. Prodrugs are variations or derivatives of the compounds of this invention which have groups cleavable under metabolic conditions. Prodrugs become the compounds of the invention which are pharmaceutically active in vivo, when they undergo solvolysis under physiological conditions or undergo enzymatic degradation. Prodrug compounds of this invention may be called single, double, triple etc., depending on the number of biotransformation steps required to release the active drug within the organism, and indicating the number of functionalities present in a precursor-type form. Prodrug forms often offer advantages of solubility, tissue compatibility, or delayed release in the mammalian organism (see, Bundgard, Design of Prodrugs, pp. 7-9, 21-24, Elsevier, Amsterdam 1985 and Silverman, The Organic Chemistry of Drug Design and Drug Action, pp. 352-401, Academic Press, San Diego, Calif., 1992). Prodrugs commonly known in the art include acid derivatives well known to practitioners of the art, such as, for example, esters prepared by reaction of the parent acids with a suitable alcohol, or amides prepared by reaction of the parent acid compound with an amine, or basic groups reacted to form an acylated base derivative. Moreover, the prodrug derivatives of this invention may be combined with other features herein taught to enhance bioavailability. The preparation of pharmaceutically acceptable isomers, solvates or hydrates would be apparent to one of ordinary skill in the art.

Glossary

Co-processing: Pharmaceutical co-processing refers to any of several possible methods in which two or more substances or compositions are combined in a single process to produce a single, combined product or composition. One common method of co-processing is spray drying a solution and/or slurry of two or more substances to produce a single product comprised of the two or more starting substances. Another common method of co-processing is to granulate a mixture of two or more substances or other starting compositions to form a single granular product incorporating the starting materials.

Cushioning Beads™: Cushioning Beads™ are spherical or semi-spherical agglomerates of suitable composition, structure and deformation property such that when present in suitable proportion in admixture with membrane coated active-loaded beads and the admixture compressed to form a pharmaceutical tablet, the cushioning beads deform preferentially (that is, they deform at lower pressures) to substantially prevent rupture or cracking of the membrane of the active-loaded beads. Generally, cushioning beads do not contain a biologically active substance. Aulton et al. [Drug Development and Industrial Pharmacy, Vol. 20, pp. 3069-3104 (1994), at page 3094] refers to them as ‘placebo millispheres.’ Mount et al. [Drug Development and Industrial Pharmacy, Vol. 22,pp.609-612 (1996), at page 612] refers to them a ‘cushioning agents.’ In addition, the definition of cushioning beads is expanded to include those active-loaded beads co-processed with cushioning components under current invention as demonstrated in FIG. 1.

Active-loaded beads: Active-loaded beads are beads (or pellets) comprised of (a) one or more biologically active substances within or as part of a core seed around which a one or more suitable functional and/or non-functional coating are applied, or (b) a core seed coated with one or more layers of biologically active substance(s) around which a one or more suitable functional and/or non-functional coating are applied. Non-functional coatings are well described in the pharmaceutical literature and may, among others, be used to mask taste, separate biologically active component layers, and as protective over-coats or under-coats for functional coatings. Functional coatings are well described in the pharmaceutical literature and may, among others, be used to delay drug release or to provide extended, sustained or prolonged release, or pulsed release. Functional and non-functional coatings have been described in numerous writings [see, for example, J. W. McGinity, Aqueous Polymeric Coatings for Pharmaceutical Dosage Forms, Marcel Dekker, New York, N.Y., 1988).

Pharmaceutical composition: A pharmaceutical composition is a designed pharmaceutical formulation assembled (processed) in such a way as to meet certain functional criteria (e.g. appropriate drug release characteristics, stability, manufacturability, patient acceptability, content uniformity). Biologically active substances are seldom administered alone, but rather as part of a pharmaceutical composition or formulation in combination with one or more non-medical ingredients called excipients that serve varied and specialized functions, such as fillers, binders, lubricants, glidants, inert core beads, release rate-controlling components, stabilizers, flavors, colors, and others. The selection of excipients and their levels in the formulation, the method of assembly, and the appropriate adjustment of process variables together determine how closely the pharmaceutical composition meets its design criteria.

Granule: Aggregates of particles obtained by wet or dry granulation processes.

LOD: Loss on drying is defined as the percentage of water removed when a material is dried in an oven, or under infrared light, with or without the aid of a vacuum

Cushioning Component: The cushioning component consists of a highly-compactable filler, such as microcrystalline cellulose, in combination with a highly water-absorbing material, such as Ac-Di-Sol®R. Disintegrants and superdisintegrants, such as starch, croscarmellose sodium, crospovidone, and sodium starch glycolate, or hydrophilic materials, such as hydroxypropyl cellulose, can be used as the highly water-absorbing material

Patient: a mammal, preferably a human, in need of treatment of a condition, disorder or disease.

Treat and Treatment: Refer to both therapeutic treatments and prophylactic or preventative measures, wherein the object is to prevent or slow down (lessen) an undesired physiological condition, disorder or disease or obtain beneficial or desired clinical results. For purposes of this invention, beneficial or desired clinical results include but are not limited to, alleviation of symptoms; diminishment of extent of condition, disorder or disease; stabilized (i.e. not worsening) state of condition, disorder or disease; delay or slowing of condition, disorder or disease progression; amelioration of the condition, disorder or disease state; remission (whether partial or total), whether detectable or undetectable; or enhancement or improvement of condition, disorder or disease. Treatment includes eliciting a cellular response that is clinically significant, without excessive levels of side effects. Treatment also includes prolonging survival as compared to expected survival if not receiving treatment.

Mammal: Refers to any animal classified as a mammal, including humans, domestic and farm animals, and zoo, sports and pet companion animals such as household pet and other domesticated animals such as, but not limited to, cattle, sheep, ferrets, swine, horses, poultry, rabbits, goats, dogs, cats and the like. Preferred companion animals are dogs and cats. Preferably, the mammal is human.

Biological property: for the purposes herein means an in vivo effector or antigenic function or activity that is directly or indirectly performed by a compound of this invention that are often shown by in vitro assays. Effector functions include receptor or ligand binding, any enzyme activity or enzyme modulatory activity, any carrier binding activity, any hormonal activity, any activity in promoting or inhibiting adhesion of cells to an extracellular matrix or cell surface molecules, or any structural role. Antigenic functions include possession of an epitope or antigenic site that is capable of reacting with antibodies raised against it.

Active Pharmaceutical Ingredient: The biologically active ingredient in any pharmaceutical composition. The “API” is the ingredient that creates the desired biological property in the patient in need of treatment.

Pharmaceutically Acceptable Salts: includes salts of compounds derived from the combination of a compound and an organic or inorganic acid. These compounds are useful in both free base and salt form. In practice, the use of the salt form amounts to use of the base form; both acid and base addition salts are within the scope of the present invention.

Therapeutic Applications

The described drug delivery technology would be applicable for any active pharmaceutical ingredient of choice wherein the preferred route of administration is an oral solution.

### FIGURES

FIG. 1: A Cut-away Schematic representing the active-loaded bead inside the cushioning bead.

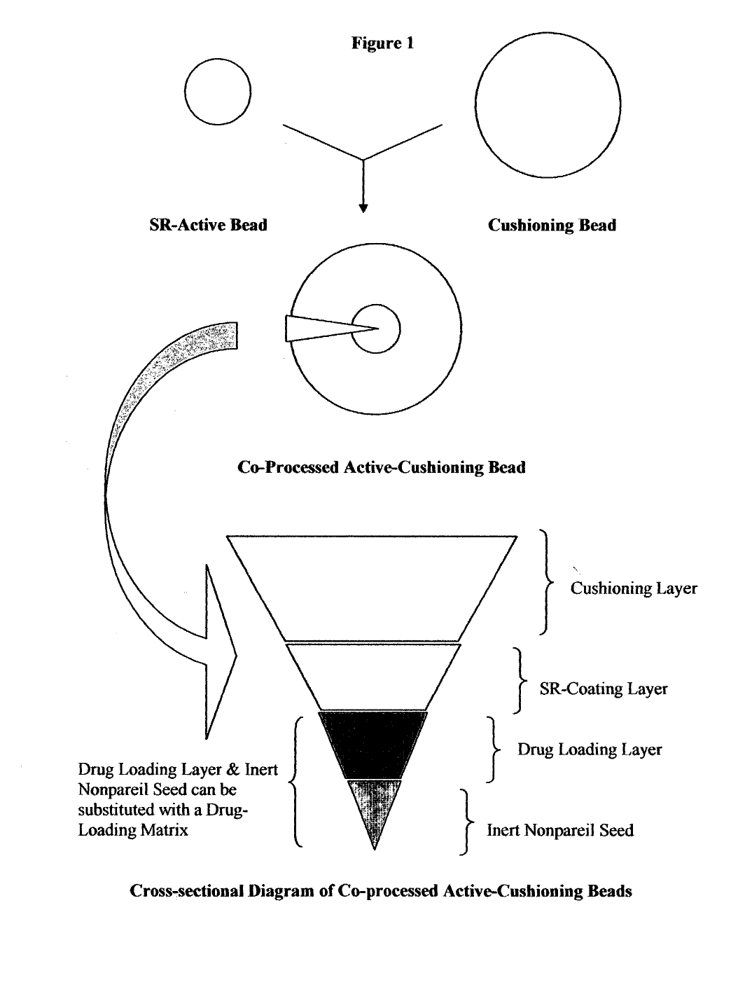

FIG. 2: A flow-chart of the manufacturing process.

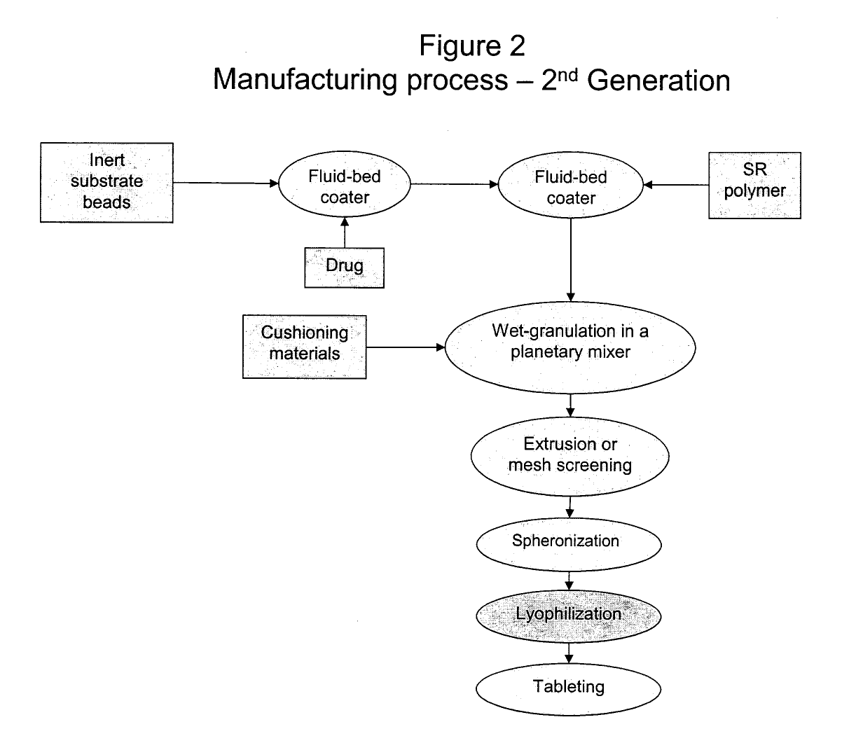

FIG. 3: Percent dissolved profile.

Table 1: Raw data dissolution.

Table 2: Relative percent dissolution.

### EXAMPLES

The examples below are intended for illustration only and not to limit the invention.

### Example 1

### Prednisolone Sodium Phosphate

Phase I—Manufacture of Active-Loaded Beads

The manufacture of prednisolone sodium phosphate-loaded beads involves a conventional drug-layering process where the active drug is dissolved in an aqueous dispersion of Opadry® clear. The dispersion is then sprayed onto the non-pareil seeds (Celpheres®) on a fluid-bed processor equipped with a Wurster column. Subsequently, the dispersion of sustained-release coating dispersion and the protective coating dispersion were sprayed onto the active-loaded non-pareil seeds in a similar manner described above.

Formulation of Dru-Layerin® Dispersion

| | |
|---|---|
| Prednisolone Sodium Phosphate | 12.0% |
| Opadry ® Clear | 2.0% |
| Purified Water | q.s. |

Formulation of Sustained-Release Coating Dispersion

| | |
|---|---|
| Eudragit ® NE-30D | 33.3% |
| Talc | 10.0% |
| Purified Water | q.s. |

Formulation of Protection Coating Dispersion

| | |
|---|---|
| Opadry ® II | 12.0% |
| Purified Water | q.s. |

Phase II—Co-Processing of Active-Loaded Beads with the Cushioning Components

The cushioning components (see below) are dry-blended and then granulated in a planetary mixer via a typical low-shear wet granulation process with purified water as a granulating fluid. Sustained-release coated active-loaded beads are added to the moistened granules subsequently. The active-loaded beads can alternatively be added to the cushioning components blend prior to the wet granulation step. The moistened granules of the well dispersed, active-loaded beads in the cushioning components are either passed through screen of appropriate size or extruded and spheronized. The beads or pellets thus obtained are then freeze-dried.

Formulation of Co-Processed Active-Loaded Beads with Cushioning Components

| | |
|---|---|
| Prednisolone Sodium Phosphate SR-coated Beads | 12.0% |
| Microcrystalline Cellulose (Avicel ® PH101) | 61.5% |
| Croscarmellose Sodium (Ac-Di-Sol ®) | 13.5% |
| Purified Water | q.s. |

Manufacturing Procedure

1. Wet mass the Avicel® PH101 and Ac-Di-Sol® in a low-shear mixer using the Purified Water as granulating fluid until a homogeneous moistened granule mass is obtained

2. Add the Prednisolone Phosphate SR-coated beads to the moistened granule mass in the mixer, and mix until a homogeneous dispersion of active beads is obtained

3. Discharge the moistened granule mass and pass it through a screen with appropriate mesh size. The granule mass is spheronized to produce spheroids/beads.

4. Freeze-dry the spheroids

Phase III—Compression of the Cushioning Beads

The final freeze-dried spheroids of the co-processed active-loaded beads with cushioning components are compressed into tablets on a rotary tablet press or equivalent.

Results and Discussion

Preliminary dissolution data using a non-validated UV assay method were obtained on the samples as noted below.

Samples Prepared and Evaluated

| Sample | Weight | Compression Force |
|---|---|---|
| Tablet 1 | 1.024 g | 5.6 MPa |
| Tablet 2 | 1.024 g | 10.3 MPa |
| Loose Granulation | 1.026 g | NA |
| (Control) | | |
| Triturated Granulation | 1.026 g | NA |

Tablets 1 and 2 were manufactured by hand operating an instrumented Stokes B-2 rotary tablet press. Voltages read off the PC are converted to force using the current calibration curve and then converted to pressure based on the punch diameter (11/16″). The granulation was preweighed and hand-filled into the die. The tablets were made one-at-a-time. The weights reported above represent the actual weights of the finished tablets.

Dissolution was carried out by means of continuous flow through a 1 cm path length cell using a Van Kel (VK 7000) dissolution system, Van Kel integrated water bath (37°) and Shimadzu UV 160U spectrophotomer fitted with cell changer. The dissolution fluid was 900 mL 0.1N HCl. USP Method 2 (paddles) rotating at 50 RPM was employed. Absorbance was read at 246 nm every 30 minutes for 12 hours.

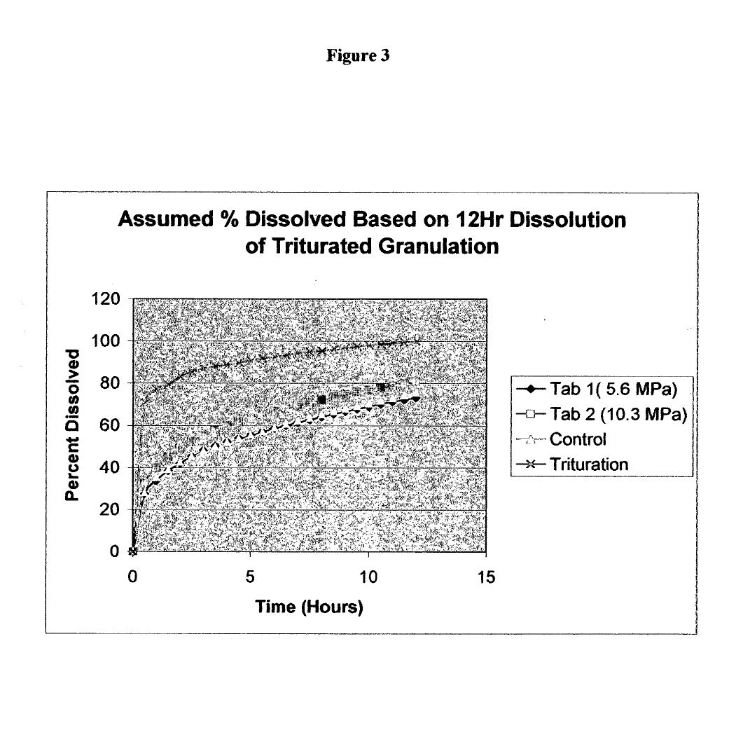

Dissolution of the triturated granules was used to get an estimate of 100% dissolution. The percent dissolved of the two tablets and the loose granules control were estimated based on the percent dissolved of the trituration after 12 hours of running. The raw data were shown in Table 1. The relative (i.e., tablets vs granules) percent dissolved were shown in Table 2. The assumed percent dissolved profiles are displayed in FIG. 3.

Similarity metrics (f2) were used to compare the similarity of the Control and Tablets 1 and 2. The results based on comparison at 12 equi-spaced (hourly) time points from 1 to 12 hours are as follows:

| Comparison | f₂ |
|---|---|
| Tab 1 (5.6 MPa) vs Control | 89.3 |
| Tab 1 (5.6 MPa) vs Tab 2 | 55.8 |
| (10.3 MPa) | |

These data strongly suggest that the cushioning system is working. The degree of similarity between Tablet 1 (5.6 MPa) and the loose, uncompressed granulation (Control) is extremely high, with an f2 of 89.3. Compression to 10.4 MPa has obviously caused some damage to the SR beads as evidenced by the more rapid dissolution of the drug from Tablet 2. Nevertheless, using FDA's criterion for similarity, (f2=or >50 indicates similarity), the dissolution profiles of Tablets 1 and 2 would still be considered similar for regulatory purposes.

Tablet 1 and Tablet 2 dissolution profiles are quite linear in time in the range from 1 to 12 hours, with correlation coefficients against time of 0.9797 and 0.9733, respectively.

TABLE 1
Dissolution Based on Absorbance
Readings

| Time | Tab 1 | Tab 2 | | |
|---|---|---|---|---|
| (Hrs) | 5.6 MPa | 10.3 MPA | Control | Trituration |
| 0 | 0 | 0 | 0.002 | 0 |
| 0.5 | 0.245 | 0.289 | 0.251 | 0.631 |
| 1 | 0.301 | 0.354 | 0.316 | 0.69 |
| 1.5 | 0.343 | 0.401 | 0.35 | 0.707 |
| 2 | 0.375 | 0.437 | 0.382 | 0.743 |
| 2.5 | 0.404 | 0.467 | 0.406 | 0.761 |
| 3 | 0.429 | 0.493 | 0.43 | 0.778 |
| 3.5 | 0.448 | 0.515 | 0.447 | 0.785 |
| 4 | 0.466 | 0.534 | 0.466 | 0.792 |
| 4.5 | 0.481 | 0.55 | 0.482 | 0.801 |
| 5 | 0.495 | 0.566 | 0.498 | 0.81 |
| 5.5 | 0.509 | 0.581 | 0.512 | 0.819 |
| 6 | 0.523 | 0.594 | 0.528 | 0.824 |
| 6.5 | 0.535 | 0.608 | 0.543 | 0.832 |
| 7 | 0.548 | 0.619 | 0.556 | 0.84 |
| 7.5 | 0.559 | 0.632 | 0.569 | 0.846 |
| 8 | 0.573 | 0.643 | 0.582 | 0.85 |
| 8.5 | 0.583 | 0.655 | 0.596 | 0.857 |
| 9 | 0.594 | 0.665 | 0.607 | 0.862 |
| 9.5 | 0.605 | 0.676 | 0.619 | 0.868 |
| 10 | 0.615 | 0.685 | 0.63 | 0.874 |
| 10.5 | 0.626 | 0.695 | 0.642 | 0.879 |
| 11 | 0.636 | 0.703 | 0.652 | 0.883 |
| 11.5 | 0.644 | 0.712 | 0.663 | 0.887 |
| 12 | 0.653 | 0.721 | 0.675 | 0.892 |

TABLE 2
Assumed % Dissolved Based on 12 hr
Dissolution of Triturated Granulation

| Time | Tab 1 | Tab 2 | | |
|---|---|---|---|---|
| (Hrs) | 5.6 MPa | 10.3 MPa | Control | Trituration |
| 0 | 0 | 0.0 | 0.2 | 0.0 |
| 0.5 | 27.5 | 32.4 | 28.1 | 70.7 |
| 1 | 33.7 | 39.7 | 35.4 | 77.4 |
| 1.5 | 38.5 | 45.0 | 39.2 | 79.3 |
| 2 | 42.0 | 49.0 | 42.8 | 83.3 |
| 2.5 | 45.3 | 52.4 | 45.5 | 85.3 |
| 3 | 48.1 | 55.3 | 48.2 | 87.2 |
| 3.5 | 50.2 | 57.7 | 50.1 | 88.0 |
| 4 | 52.2 | 59.9 | 52.2 | 88.8 |
| 4.5 | 53.9 | 61.7 | 54.0 | 89.8 |
| 5 | 55.5 | 63.5 | 55.8 | 90.8 |
| 5.5 | 57.1 | 65.1 | 57.4 | 91.8 |
| 6 | 58.6 | 66.6 | 59.2 | 92.4 |
| 6.5 | 60.0 | 68.2 | 60.9 | 93.3 |
| 7 | 61.4 | 69.4 | 62.3 | 94.2 |
| 7.5 | 62.7 | 70.9 | 63.8 | 94.8 |
| 8 | 64.2 | 72.1 | 65.2 | 95.3 |
| 8.5 | 65.4 | 73.4 | 66.8 | 96.1 |
| 9 | 66.6 | 74.6 | 68.0 | 96.6 |
| 9.5 | 67.8 | 75.8 | 69.4 | 97.3 |
| 10 | 68.9 | 76.8 | 70.6 | 98.0 |
| 10.5 | 70.2 | 77.9 | 72.0 | 98.5 |
| 11 | 71.3 | 78.8 | 73.1 | 99.0 |
| 11.5 | 72.2 | 79.8 | 74.3 | 99.4 |
| 12 | 73.2 | 80.8 | 75.7 | 100.0 |

We claim the following:

1. A method for the manufacture of Cushioning Beads™ that includes:
a) blending of a highly-compactable filler in combination with a highly water-absorbing material and;
b) adding purified water to the mixture of highly-compactable filler and highly water-absorbing material until granules are formed by visual inspection thus creating the cushioning component and;
c) adding active-loaded beads to the cushioning component to create a mixture followed by an optional step of extrusion and spheronization and;
d) freeze-drying of the mixture of active-loaded beads and cushioning component with or without said extrusion and spheronization to create the Cushioning Beads™; and
e) compressing the Cushioning Beads™ into a tablet for treatment of a patient in need of said treatment.

2. A method for the manufacture of Cushioning Beads™ that includes:
a) blending of Avicel® PH101 and Ac-Di-Sol® and;
b) adding purified water to the mixture of Avicel® PH101 and Ac-Di-Sol® until granules are formed by visual inspection thus creating the cushioning component and;
c) adding of active-loaded beads to the cushioning component to create a mixture followed by an optional step of extrusion and spheronization and;
d) freeze-drying of the mixture of active-loaded beads and cushioning component with or without said extrusion and spheronization to create the Cushioning Beads™; and
e) compressing the Cushioning Beads™ into a tablet for treatment of a patient in need of said treatment.

3. A method for the manufacture of Cushioning Beads™ that includes:
a) blending of Avicel® PH101 and Ac-Di-Sol® in a ratio that the mixture will have Ac-Di-Sol ranging from 5 to 90% by weight and;
b) adding of purified water to the mixture of Avicel® PH101 and Ac-Di-Sol® until granules are formed by visual inspection thus creating the cushioning component and;
c) adding of active-loaded beads to the cushioning component to create a mixture followed by an optional step of extrusion and spheronization and;
d) freeze-drying of the mixture of active-loaded beads and the cushioning component with or without said extrusion and spheronization to create the Cushioning Beads™; and
e) compressing the Cushioning Beads™ into a tablet for treatment of a patient in need of said treatment.

4. A method for the manufacture of Cushioning Beads™ that includes:
a) blending of a highly-compactable filler in combination with a highly water-absorbing material and;
b) adding of purified water to the mixture of highly-compactable filler and highly water-absorbing material until granules are formed by visual inspection thus creating the cushioning component and;
c) adding of active-loaded beads to the cushioning component to create a mixture followed by an optional step of extrusion and spheronization and;
d) freeze-drying of the mixture of active-loaded beads and the cushioning component with or without said extrusion and spheronization until a LOD of 2-15% is achieved to create the Cushioning Beads™ followed by an optional step of extrusion and spheronization of the Cushioning Beads™; and
e) compressing the Cushioning Beads™ into a tablet for treatment of a patient in need of said treatment.

5. A method for the manufacture of Cushioning Beads™ that includes:
a) blending of Avicel® PH101 and Ac-Di-Sol® and;
b) adding purified water to the mixture of Avicel® PH101 and Ac-Di-Sol® until granules are formed by visual inspection thus creating the cushioning component and;
c) adding of active-loaded beads to the cushioning component to create a mixture followed by an optional step of extrusion and spheronization and;
d) freeze-drying of the mixture of active-loaded beads and cushioning component with or without said extrusion and spheronization until a LOD of 2-15% is achieved to create the Cushioning Beads™; and
e) compressing the Cushioning Beads™ into a tablet for treatment of a patient in need of said treatment.

6. A method for the manufacture of Cushioning Beads™ that includes:
a) blending of Avicel® PH101 and Ac-Di-Sol® in a ratio that the mixture will have Ac-Di-Sol ranging from 5 to 90% by weight;
b) adding of purified water to the mixture of Avicel® PH101 and Ac-Di-Sol® until granules are formed by visual inspection thus creating the cushioning component and;
c) adding of active-loaded beads to the cushioning component to create a mixture followed by an optional step of extrusion and spheronization and;
d) freeze-drying of the mixture of active-loaded beads and cushioning component with or without said extrusion and spheronization until a LOD of 2-15% is achieved to create the Cushioning Beads™; and
e) compressing the Cushioning Beads™ into a tablet for treatment of a patient in need of said treatment.

7. A method for the manufacture of Cushioning Beads™ that includes:
a) blending of a highly-compactable filler in combination with a highly water-absorbing material and;
b) adding purified water to the mixture of highly-compactable filler and highly water-absorbing material until granules are formed by visual inspection thus creating the cushioning component and;
c) adding active-loaded beads to the cushioning component to create a mixture followed by an optional step of extrusion and spheronization and;
d) freeze-drying of the mixture of active-loaded beads and cushioning component with or without said extrusion and spheronization to create the Cushioning Beads™; and
e) encapsulating the Cushioning Beads™ into a capsule for treatment of a patient in need of said treatment.

8. A method for manufacture of Cushioning Beads™ that includes:
a) blending of Avicel® PH101 and Ac-Di-Sol® and;
b) adding purified water to the mixture of Avicel® PH101 and Ac-Di-Sol® until granules are formed by visual inspection thus creating the cushioning component and;
c) adding of active-loaded beads to the cushioning component to create a mixture followed by an optional step of extrusion and spheronization and;
d) freeze-drying of the mixture of active-loaded beads and cushioning component with or without said extrusion and spheronization to create the Cushioning Beads™; and
e) encapsulating the Cushioning Beads™ into a capsule for treatment of a patient in need of said treatment.

9. A method for the manufacture of Cushioning Beads™ that includes:
a) blending of Avicel® PH101 and Ac-Di-Sol® in a ratio that the mixture will have Ac-Di-Sol ranging from 5 to 90% by weight and;
b) adding of purified water to the mixture of Avicel® PH101 and Ac-Di-Sol® until granules are formed by visual inspection thus creating the cushioning component and;
c) adding of active-loaded beads to the cushioning component to create a mixture followed by an optional step of extrusion and spheronization and;
d) freeze-drying of the mixture of active-loaded beads and the cushioning component with or without said extrusion and spheronization to create the Cushioning Beads™; and
e) encapsulating the Cushioning Beads™ into a capsule for treatment of a patient in need of said treatment.

10. A method for the manufacture of Cushioning Beads™ that includes:
a) blending of a of a highly-compactable filler in combination with a highly water-absorbing material and;
b) adding of purified water to the mixture of highly-compactable filler and highly water-absorbing material until granules are formed by visual inspection thus creating the cushioning component and;
c) adding of active-loaded beads to the cushioning component to create a mixture followed by an optional step of extrusion and spheronization and;
d) freeze-drying of the mixture of active-loaded beads and the cushioning component with or without said extrusion and spheronization until a LOD of 2-15% is achieved to create the Cushioning Beads™; and
e) encapsulating the Cushioning Beads™ into a capsule for treatment of a patient in need of said treatment.

11. A method for the manufacture of Cushioning Beads™ that includes:
a) blending of Avicel®R PH101 and Ac-Di-Sol® and;
b) adding purified water to the mixture of Avicel® PH101 and Ac-Di-Sol® until granules are formed by visual inspection thus creating the cushioning component and;
c) adding of active-loaded beads to the cushioning component to create a mixture followed by an optional step of extrusion and spheronization and;
d) freeze-drying of the mixture of active-loaded beads and cushioning component with or without said extrusion and spheronziation until a LOD of 2-15% is achieved to create the Cushioning Beads™; and
e) encapsulating the Cushioning Beads™ into a capsule for treatment of a patient in need of said treatment.

12. A method for the manufacture of Cushioning Beads™ that includes:
a) blending of Avicel® PH101 and Ac-Di-Sol® in a ratio that the mixture will have Ac-Di-Sol ranging from 5 to 90% by weight and;
b) adding of purified water to the mixture of Avicel® PH101 and Ac-Di-Sol® until granules are formed by visual inspection thus creating the cushioning component and;
c) adding of active-loaded beads to the cushioning component to create a mixture followed by an optional step of extrusion and spheronization and;
d) freeze-drying of the mixture of active-loaded beads and cushioning component with or without said extrusion and spheronization until a LOD of 2-15% is achieved to create the Cushioning Beads™; and
e) encapsulating the Cushioning Beads™ into a capsule for treatment of a patient in need of said treatment.

13. A method for the manufacture of Cushioning Beads™ that includes:
a) blending of a highly-compactable filler in combination with a highly water-absorbing material and;
b) adding purified water to the mixture of highly-compactable filler and highly water-absorbing material until granules are formed by visual inspection thus creating the cushioning component and;
c) adding active-loaded beads to the cushioning component to create a mixture followed by an optional step of extrusion and spheronization and;
d) freeze-drying of the mixture of active-loaded beads and cushioning component with or without said extrusion and spheronization to create the Cushioning Beads™; and
c) placing the Cushioning Beads™ into a sachet that maybe added to a liquid for treatment of a patient in need of said treatment.

14. A method for manufacture of Cushioning Beads™ that includes:
a) blending of Avicel® PH101 and Ac-Di-Sol® and;
b) adding purified water to the mixture of Avicel® PH101 and Ac-Di-Sol® until granules are formed by visual inspection thus creating the cushioning component and;
c) adding of active-loaded beads to the cushioning component to create a mixture followed by an optional step of extrusion and spheronization and;
d) freeze-drying of the mixture of active-loaded beads and cushioning component with or without said extrusion and spheronization to create the Cushioning Beads™; and
e) placing the Cushioning Beads™ into a sachet for treatment of a patient in need of said treatment.

15. A method for the manufacture of Cushioning Beads™ that includes:
a) blending of Avicel® PH101 and Ac-Di-Sol® in a ratio that the mixture will have Ac-Di-Sol ranging from 5 to 90% by weight and;
b) adding of purified water to the mixture of Avicel® PH101 and Ac-Di-Sol® until granules are formed by visual inspection thus creating the cushioning component and;
c) adding of active-loaded beads to the cushioning component to create a mixture followed by an optional step of extrusion and spheronization and;
d) freeze-drying of the mixture of active-loaded beads and the cushioning component with or without said extrusion and spheronization to create the Cushioning Beads™; and
e) placing the Cushioning Beads™ into a sachet for treatment of a patient in need of said treatment.

16. A method for the manufacture of Cushioning Beads™ that includes:
a) blending of a highly-compactable filler in combination with a highly water-absorbing material and;
b) adding of purified water to the mixture of highly-compactable filler and highly water-absorbing material until granules are formed by visual inspection thus creating the cushioning component and;
c) adding of active-loaded beads to the cushioning component to create a mixture followed by an optional step of extrusion and spheronization and;
d) freeze-drying of the mixture of active-loaded beads and the cushioning component with or without said extrusion and spheronization until a LOD of 2-15% is achieved to create the Cushioning Beads; and
e) placing the Cushioning Beads™ into a sachet for treatment of a patient in need of said treatment.

17. A method for the manufacture of Cushioning Beads™ that includes:
a) blending of Avicel® PH101 and Ac-Di-Sol® and;
b) adding purified water to the mixture of Avicel® PH101 and Ac-Di-Sol® until granules are formed by visual inspection thus creating the cushioning component and;
c) adding of active-loaded beads to the cushioning component to create a mixture followed by an optional step of extrusion and spheronization and;
d) freeze-drying of the mixture of active-loaded beads and cushioning component with or without said extrusion and spheronization until a LOD of 2-15% is achieved to create the Cushioning Beads™; and
e) placing the Cushioning Beads™ into a sachet for treatment of a patient in need of said treatment.

18. A method for the manufacture of Cushioning Beads™ that includes:
a) blending of Avicel® PH101 and Ac-Di-Sol® in a ratio that the mixture will have Ac-Di-Sol ranging from 5 to 90% by weight and;
b) adding of purified water to the mixture of Avicel® PH101 and Ac-Di-Sol® until granules are formed by visual inspection thus creating the cushioning component and;
c) adding of active-loaded beads to the cushioning component to create a mixture followed by an optional step of extrusion and spheronization and;
d) freeze-drying of the mixture of active-loaded beads and cushioning component with or without said extrusion and spheronization until a LOD of 2-15% is achieved to create the Cushioning Beads™; and
e) placing the Cushioning Beads™ into a sachet for treatment of a patient in need of said treatment.

19. A method for the manufacture of Cushioning Beads™ that includes:
a) blending of a highly-compactable filler in combination with a highly water-absorbing material and;
b) adding purified water to the mixture of highly-compactable filler and highly water-absorbing material until granules are formed by visual inspection thus creating the cushioning component and;
c) adding more than one type of active-loaded beads to the cushioning component to create a mixture followed by an optional step of extrusion and spheronization and;
d) freeze-drying of the mixture of active-loaded beads and cushioning component with or without said extrusion and spheronization to create the Cushioning Beads™; and
e) compressing the Cushioning Beads™ into a tablet for treatment of a patient in need of said treatment.

20. A method for the manufacture of Cushioning Beads™ that includes:
a) blending of Avicel® PH101 and Ac-Di-Sol® and;
b) adding purified water to the mixture of Avicel® PH101 and Ac-Di-Sol® until granules are formed by visual inspection thus creating the cushioning component and;
c) adding of more than one type of active-loaded beads to the cushioning component to create a mixture followed by an optional step of extrusion and spheronization and;
d) freeze-drying of the mixture of active-loaded beads and cushioning component with or without said extrusion and spheronization to create the Cushioning Beads™; and
e) compressing the Cushioning Beads™ into a tablet for treatment of a patient in need of said treatment.

21. A method for the manufacture of Cushioning Beads™ that includes:
a) blending of Avicel® PH101 and Ac-Di-Sol® in a ratio that the mixture will have Ac-Di-Sol ranging from 5 to 90% by weight and;
b) adding of purified water to the mixture of Avicel® PH101 and Ac-Di-Sol® until granules are formed by visual inspection thus creating the cushioning component and;
c) adding of more than one type of active-loaded beads to the cushioning component to create a mixture followed by an optional step of extrusion and spheronization and;
d) freeze-drying of the mixture of active-loaded beads and the cushioning component with or without said extrusion and spheronization to create the Cushioning Beads™; and
e) compressing the Cushioning Beads™ into a tablet for treatment of a patient in need of said treatment.

22. A method for the manufacture of Cushioning Beads™ that includes:
a) blending of a highly-compactable filler in combination with a highly water-absorbing material and;
b) adding of purified water to the mixture of highly-compactable filler and highly water-absorbing material until granules are formed by visual inspection thus creating the cushioning component and;
c) adding of more than one type of active-loaded beads to the cushioning component to create a mixture followed by an optional step of extrusion and spheronization and;
d) freeze-drying of the mixture of active-loaded beads and the cushioning component with or without said extrusion and spheronization until a LOD of 2-15% is achieved to create the Cushioning Beads™; and
e) compressing the Cushioning Beads™ into a tablet for treatment of a patient in need of said treatment.

23. A method for the of manufacture of Cushioning Beads™ that includes:
a) blending of Avicel® PH101 and Ac-Di-Sol® and;
b) adding purified water to the mixture of Avicel® PH101 and Ac-Di-Sol®) until granules are formed by visual inspection thus creating the cushioning component and;
c) adding of more than one type of active-loaded beads to the cushioning component to create a mixture followed by an optional step of extrusion and spheronization and;
d) freeze-drying of the mixture of active-loaded beads and cushioning component with or without said extrusion and spheronization until a LOD of 2-15% is achieved to create the Cushioning Beads™; and
e) and compressing the Cushioning Beads™ into a tablet for treatment of a patient in need of said treatment.

24. A method for the manufacture of Cushioning Beads™ that includes:
a) blending of Avicel® PH101 and Ac-Di-Sol® in a ratio that the mixture will have Ac-Di-Sol ranging from 5 to 90% by weight and;
b) adding of purified water to the mixture of Avicel® PH101 and Ac-Di-Sol® until granules are formed by visual inspection thus creating the cushioning component and;
c) adding of more than one type of active-loaded beads to the cushioning component to create a mixture followed by an optional step of extrusion and spheronization and;
d) freeze-drying of the mixture of active-loaded beads and cushioning component with or without said extrusion and spheronization until a LOD of 2-15% is achieved to create the Cushioning Beads™; and
e) compressing the Cushioning Beads™ into a tablet for treatment of a patient in need of said treatment.
